(19) United States
(12) Patent Application Publication
(10) Pub. No.: US 2007/0203567 A1
(43) Pub. Date: Aug. 30, 2007

(54) Bifurcation aneurysm treatment stent with distal web

(76) Inventor: Elad I. Levy, Amherst, NY (US)

Correspondence Address: Donald N. Halgren, 35 Central Street, Manchester, MA, 01944, US

(21) Appl. No.: 11/645,248

(22) Filed: Dec. 23, 2006

Related U.S. Application Data
(60) Provisional application No. 60/755,639, filed on Dec. 31, 2005.
(60) Provisional application No. 60/753,764, filed on Dec. 23, 2005.

(51) Int. Cl. A61F 2/90

(52) U.S. Cl. 623/1.15; 623/1.31; 623/1.36

(57) ABSTRACT

A stent device for introduction into a bifurcation aneurysm, comprising a radially expandable elongated wire mesh tube having a proximal end and a distal end. The distal end is expandable into a cone shaped portion of a larger diameter than the proximal end. A web of material is arranged across said distal end of said stent device for sealing an aneurysm at a neck portion thereof.

### BACKGROUND OF THE INVENTION

1. Field of the Invention

This invention relates to medical devices such as stents for treating aneurysms particularly bifurcation aneurysms. This non-provisional patent application is based upon Provisional Patent Application Ser. No. 60/755,639, filed Dec. 31, 2005, and upon Provisional Patent Application Ser. No. 60/753,764 filed Dec. 23, 2005, each of which are incorporated herein by reference.

2. Prior Art

Current treatment of bifurcation aneurysms currently utilize balloons and a stent. However, such balloons may at least temporarily occlude blood flow through the vessels in which they are placed. Those balloons also need to be deflated and removed at the end of a vessel remodeling session. Such balloons may also rupture an aneurysm and/or a vessel when that balloon is inflated. Utilizing a stent with the balloon in a bifurcation aneurysm does not protect both of its efferent vessels. Such balloon vessel remodeling also requires two experienced surgeons and two catheters simultaneously, in a single vessel at the same time.

It is an object of the present invention to overcome the disadvantages of the prior art.

It is a further object of the present invention to provide a bifurcation aneurysm treatment which will allow proper blood flow during the treatment procedure, and to prevent reflux of any embolic agent placed within the aneurysm.

It is a further object of the present invention to provide a novel apparatus and method for the treating and sealing of a bifurcation aneurysm.

### BRIEF SUMMARY OF THE INVENTION

The present invention relates to an elongated aneurysm treating stent device having an open proximal end and a closed distal end. The stent device in its deliverable form is cylindrically shaped and is preferably constructed from a pattern of woven metallic fibers. The proximal end of the stent device may have a plurality of radial opaque markers thereon. An elongated electrolytic tethering wire is arranged at several circumferential locations on the proximal end of the stent device. The tethering wires join a common electrolytic tethering wire which extends through a delivery catheter. The tethering wires are attached to the proximal end of the stent device that remain as electrolytic junctions. Those electrolytic junctions are arranged so as to be severed once the stent device has been put in place within an aneurysm. The webbed design of the stent device is woven so as to have larger openings between the web fibers towards the distal end of the stent device.

Further, upon expansion of the distal end into its waffle cone configuration further facilitates the larger openings of that expanded stent at its distal end.

In one preferred embodiment of the present invention, a web of foraminous or non-foraminous material is disposed across the distal end of the cone-shapable stent, so as to provide a sealing-like web across the neck of an aneurysm at a bifurcation. That web may in one embodiment, be perforable so as to permit the passage of a microwire therethrough as will be explained hereinbelow.

The stent in its unexpanded form, may have a distalmost web of sheet material thereon, or alternatively, a continuous woven pattern of cell openings, distal of the body of the stent itself. The openings or cell size of the distal web portion in its preferred format, are smaller than the cell or opening portions in the main body portion of the stent.

In a further preferred embodiment of the present invention, a plurality of anchoring struts are flayed outwardly generally spaced adjacent the distalmost portion of the stent. Those anchoring struts, somewhat J-shaped have their respective distal ends extending somewhat radially outwardly to a diameter somewhat less than the expanded diameter of the distalmost portion of those distalmost stent cells.

Those outwardly extending anchoring struts are arranged so as to engage the innermost edge of the peripheral lip of the neck of the aneurysm, at the bifurcation. Such circumferential array of anchoring struts thus prevents advancement and/or displacement of the distal end of the stent from going too far within the aneurysm itself. The waffle-shaped cone thereon nests within the circumference of the aneurysm adjacent its neck and provides a sealing web thereacross.

In delivery of such a stent within a bifurcation aneurysm, a microwire is directed through a parent vessel and into the aneurysm. A microcatheter is fed over the microwire and the microwire after several steps, is subsequently removed. The microcatheter is however temporarily left within the aneurysm at the bifurcation. The waffle cone stent is loaded through the microcatheter and is advanced over the microwire and into the aneurysm through the parent vessel. An electrolytic tethering wire is attached to the proximal most end of the waffle cone stent. The delivery catheter surrounding the waffle cone stent may then be removed proximally so as to expose the waffle cone stent device to/against the walls of the parent vessel. The distalmost end of the waffle cone stent is thus permitted to expand immediately within the neck of the aneurysm. The anchoring struts expand outwardly along with the distalmost portion of the waffle cone stent, so as to nest within the neck of the aneurysm. The microcatheter, still within the waffle cone stent, is utilized to feed an elongated embolitic member for delivery within the aneurysm itself. Such a microcatheter or embolitic material may pierce the web, or pass through one of the small cell openings in the distally located web of the waffle cone shaped stent. The result would be an embolitic member fully engulfing the aneurysm while its microcatheter delivery system is removed therefrom. The waffle cones stent with its anchoring structs expanded, would remain therewithin the aneurysm as well. The expanded cell structure of the distal end portion of the waffle cone stent would permit blood to flow through the parent vessel and across the arms of those adjoining vessels at the bifurcation.

The invention thus comprises a stent device for introduction into and for treatment of a bifurcation aneurysm, comprising a radially expandable elongated wire mesh tube having a proximal end and a distal end, the distal end being expandable into a cone shaped portion of a larger diameter than the proximal end, and a web of material arranged across the distal end of the stent device for sealing an aneurysm at a neck portion thereof. The web may be foraminous. The web may be non-foraminous (no openings). The stent device may include an arrangement of struts at or near the distal end of the stent device. The struts preferably have an aneurysm neck-portion-engaging distal end. The web may have openings therethrough which are smaller than wall openings in the proximal end of the stent device. The web may be comprised of a foldable film of polymer. The web may be radiopaque. The web is preferably nestable across a neck portion of an aneurysm to seal embolytic material therein. The web is pierceable by a microcatheter.

The invention also includes a method of treating a bifurcation aneurysm comprising one or more of the following steps which are: introducing a microwire into the aneurysm, introducing a waffle-cone-shapable microcatheter into the aneurysm over the microwire, expanding a distal end of the microcatheter into a tapered waffle-cone shape within the bifurcation aneurysm, and pivoting outwardly an annular array of anchoring struts from a circumferential array thereof spaced proximately adjacent the distal end of the catheter so as to anchor the distal end of the microcatheter within the aneurysm. The method may include arranging a pierceable web across the distal end of the microcatheter, introducing an embolitic agent through the catheter and the web on the distal end of the catheter, distally beyond the anchoring struts and into the aneurysm. The pierceable web may be foraminous. The pierceable web may be made of a non-foraminous film, to provide a sealed distal end to permit an unencumbered aneurysm-entering distal motion of the catheter. The struts preferably have an outer end of “ski-tip” shape, to facilitate anchoring the catheter within a neck portion of the aneurysm. The method may include forming the ski-tip end of the struts to extend separate and apart from the distalmost end of the microcatheter.

The invention also comprises a stent device for introduction into and for treatment of a bifurcation aneurysm, comprising: a radially expandable elongated wire mesh tube having a proximal end and a distal end, the distal end being expandable into a cone-shapeable portion of a larger diameter than its proximal end. The stent has foraminous wall portions with openings therein of a first opening size. A pierceable web of material is arranged across the distal end of the stent device for sealing an aneurysm at a neck portion thereof, the web having foraminous portions with openings therein of a second opening size. The second opening size being smaller than the openings of the first opening size. The stent device preferably as a circumferential array of radially outwardly splayable anchoring struts, the circumferential array of struts being disposed proximately adjacent a zig-zag distalmost wire wall portion of the cone-shapeable portion of the stent device. The anchoring struts have a distalmost tip portion which is preferably free of attachment to the zig-zag distalmost wire wall portion of the stent device, to permit their radial extension about the neck of an aneurysm.

### BRIEF DESCRIPTION OF THE DRAWINGS

The objects and advantages of the present invention will become more apparent when viewed in conjunction with the following drawings, in which:

FIG. 1 is a side view of a waffle cone stent within the parent vessel;

FIG. 1A is a view of the top of the stent shown in FIG. 1;

FIG. 2 is a side elevational view of a further embodiment of the stent, in an unexpanded form;

FIG. 3 is a side elevational view of the waffle cone stent with a distal web arranged thereon;

FIG. 4 is a side elevational view of the stent shown in FIG. 3, arranged within an aneurysm;

FIG. 5 is a view of a micro catheter and micro wire delivery system for a stent;

FIG. 6 is a view of the micro wire of the delivery system remaining in the aneurysm;

FIG. 7 is a view of the delivery catheter and micro wire with a stent arranged therein.

FIG. 8 is a view of a micro wire and micro catheter delivery arrangement;

FIG. 9 is a view of a waffle cone stent in its pre-expanded configuration, delivered within an aneurysm;

FIG. 10 is a view of an expanded waffle cone stent with a micro wire therethrough arranged within the aneurysm;

FIG. 11 is a view of the expanded waffle cone stent in the neck of an aneurysm shown in the delivery of an embolytic member therewithin; and

FIG. 12 is a view of the waffle cone stent expanded within the neck of an aneurysm, showing an embolytic member expanding and filling the aneurysm itself.

### DESCRIPTION OF THE PREFERRED EMBODIMENTS

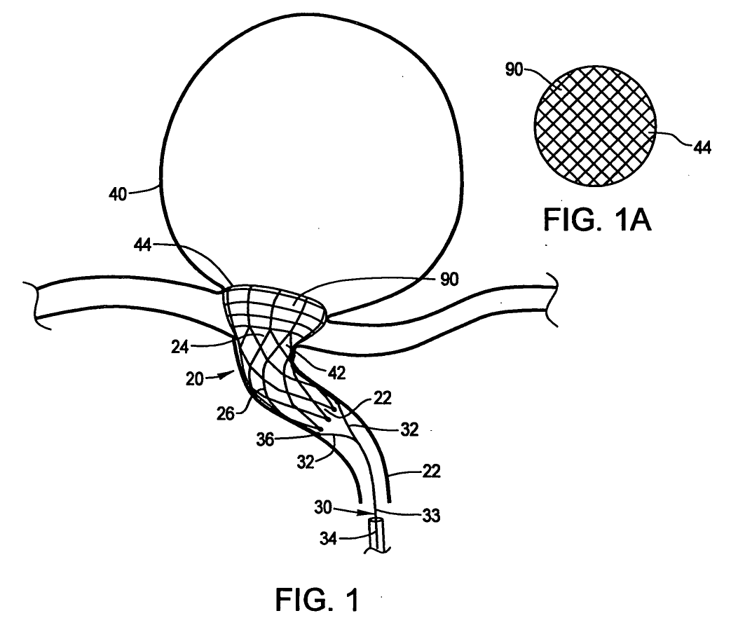

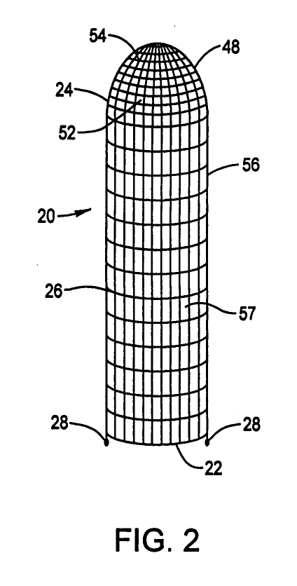

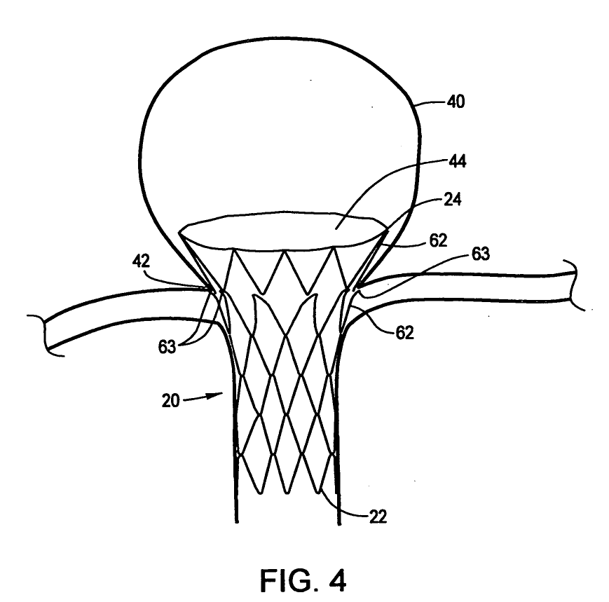

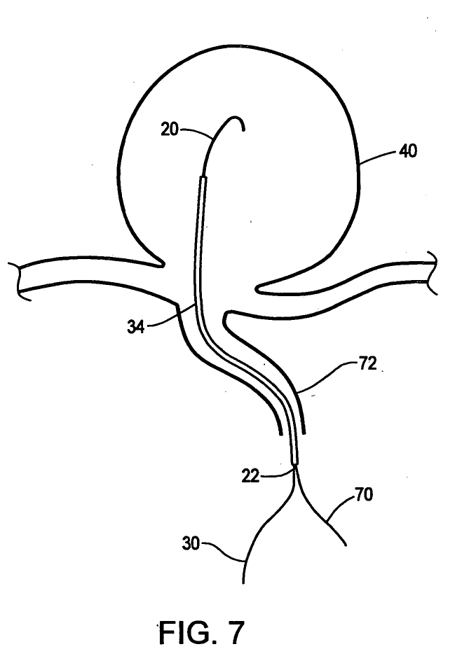

The present invention comprises an elongated aneurysm treating stent device 20 having an open proximal end 22 and a closed distal end 24, as shown in FIG. 1. The stent device 20 in its deliverable form is cylindrically shaped and is preferably constructed from a pattern of woven metallic fibers 26, as is represented in FIG. 2. The proximal end 22 of the stent device 20 may have a plurality of radial opaque markers 28 thereon. An elongated electrolytic tethering wire arrangement 30 is secured at several circumferential locations on the proximal end 22 of the stent device 20, as may be seen in FIGS. 1 and 7. The distal end of the tethering wire arrangement 30 comprises a plurality of short wires 32 which join a proximalmost common electrolytic tethering wire 33 which extends through a delivery catheter 34. The tethering wires 32 are attached to the proximal end 22 of the stent device 20 that remain as electrolytic junctions 36, as shown in FIG. 1. Those electrolytic junctions 36 are arranged so as to be severed by an electrical or mechanical severance means, not shown for clarity, once the stent device 20 has been put in place in or nesting adjacent an aneurysm 40, as is represented in FIGS. 1 and 4. The webbed design of the stent device 20 is fabricated/woven so as to have larger openings or cells 42 between the web fibers towards the distal end 24 of the stent device 20, than at its proximal end 22, as represented in the embodiments shown in FIGS. 1, 3, 4, 10, 11 and 12. Further, upon expansion of the distal end 24 into its waffle cone configuration further facilitates the larger openings of that expanded stent 20 at its distal end 24, as represented in FIG. 4.

In one preferred embodiment of the present invention, a web or film of foraminous or perforable non-foraminous material 44 is disposed across the distal end 24 of the cone-shapable stent 20, as represented in FIG. 1A, so as to provide a sealing-like web across the neck 42 of an aneurysm 40 at a vessel bifurcation as represented in FIGS. 1, and 4. That web 44 may in one embodiment, be perforable so as to permit the “piercing” passage of a microwire 50 therethrough as will be further explained hereinbelow.

The stent device 20, in its generally unexpanded form, as represented in FIG. 2 may have a non-unitary distalmost web of sheet material 48 thereon, or alternatively, a unitary continuously woven/fabricated pattern 52 of cell openings 54, distal of the generally cylindrically-shaped, main body portion 56 of the stent device 20 itself. The distal openings or cells 54 of the distal web 48 portion or the continued woven portion 52, in a preferred format, are smaller than the cell or opening portions 57 in the main body portion 56 of the stent device 20.

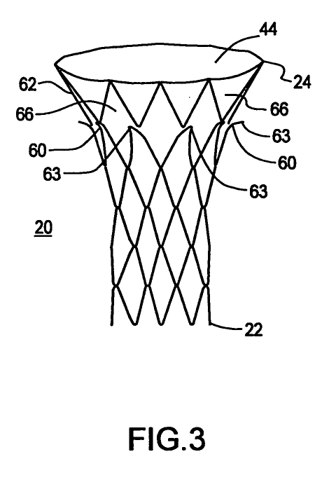

In a further preferred embodiment of the present invention represented in FIG. 3, a plurality of somewhat “ski tip” shaped displaceable anchoring struts 60 are flayed generally radially outwardly, spaced proximally adjacent the distalmost “zig-zag” configuration (in this embodiment) of distal end wires 62 or portion of the stent device 20, as is represented in FIG. 3. Those anchoring struts 60, somewhat J-shaped have their respective distal ends 63 extending somewhat radially outwardly to a diameter somewhat less than the expanded diameter of the distalmost end wires 62 of those distalmost stent cells 66.

Those outwardly extending anchoring struts 60 are arranged so as to engage the innermost edge of the inwardly directed peripheral lip of the neck 42 of the aneurysm 40, at the bifurcation, as is represented in FIG. 4. Such circumferential array of anchoring struts 62 thus prevents advancement and/or displacement of the distal end 24 of the stent device 20 from going too far within the aneurysm 40 itself. The “waffle-shaped cone” of the distal end of the stent device 20 thus is arranged to nest within the circumference of the aneurysm 40 adjacent its neck 42. The stent device 20 with its distalmost screen tent provides a sealing web 44 thereacross, as is represented in FIGS. 3 and 4.

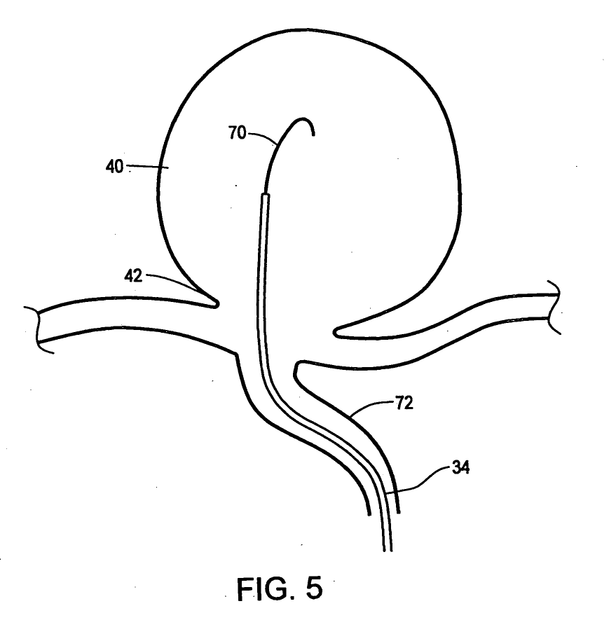

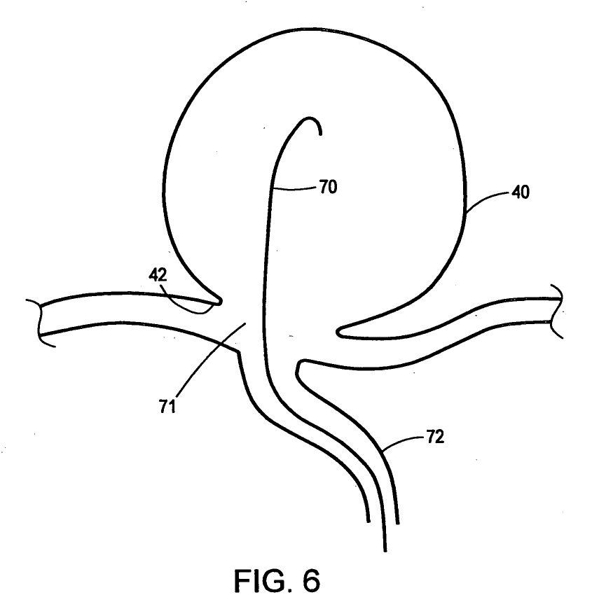

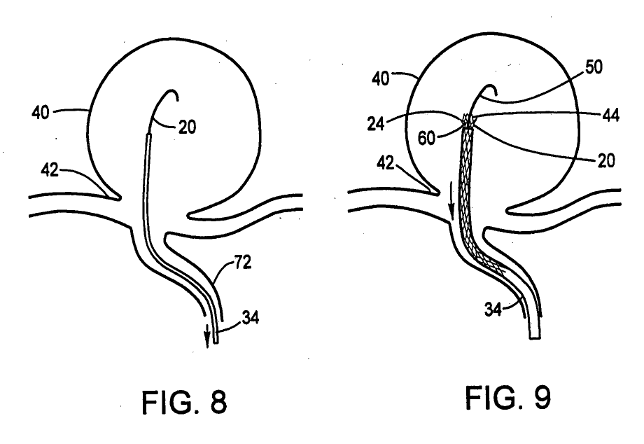

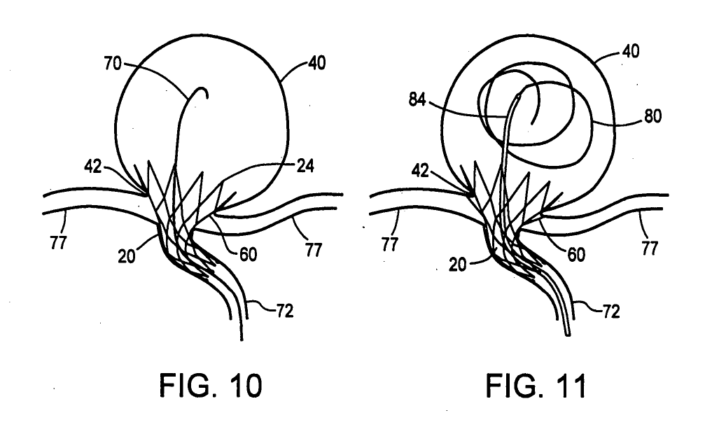

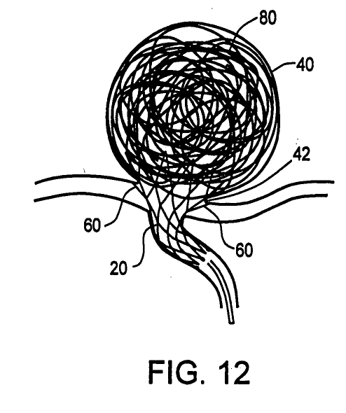

The delivery of such a stent device 20 within a bifurcation aneurysm 40, is represented in FIGS. 5 through 9, wherein a microwire 70 is directed through the parent vessel 72 and into the aneurysm 40. The microcatheter 34 is fed over the microwire 70, as is represented in FIG. 5. The microwire 70 after several steps, is however, ultimately removed. The microcatheter 34 is however temporarily left within the aneurysm 40 at the bifurcation 71, as represented in FIG. 7. The waffle cone stent is loaded through the microcatheter 34 and is advanced over the microwire 70 and into the aneurysm 40 through the parent vessel 72. The electrolytic tethering wire arrangement 30 is attached to the proximalmost end 22 of the waffle cone stent 20, as represented in FIG. 7. The delivery catheter 34 surrounding the waffle cone stent device 20 may then be removed proximally, as suggested by the representation in FIG. 8, so as to expose the waffle cone stent device 20 to/against the walls of the parent vessel 72, as represented in FIG. 9. The distalmost end 24 of the waffle cone stent device 20 is thus permitted to expand immediately within the neck 42 of the aneurysm 40, as represented in FIG. 10. The anchoring struts 60 expand radially outwardly along with the distalmost portion of the waffle cone stent device 20, as represented in FIGS. 4 and 11, so as to nest within the neck 42 of the aneurysm 40, as represented in FIGS. 4, 10, 11 and 12. A microcatheter 84, represented in FIG. 11, may be introduced over the microwire 70 within the waffle cone stent device 20, and is utilized to feed an elongated embolytic member 80 for delivery into the aneurysm 40 itself, as is represented in FIGS. 11 and 12. Such a microcatheter or embolytic material may in one embodiment, pierce the web or film 44, or pass through one of the small cell openings 90 in the distally located web 44 of the waffle cone shaped stent device 20, initially represented in FIG. 1A. The result would be an embolytic member fully engulfing the aneurysm 40 while its microcatheter delivery system 34 is removed therefrom. The waffle cones stent device 20 with its anchoring structs expanded 60, would remain secured within the aneurysm 40 as well. The expanded open cell structure of the distal end portion 24 of the waffle cone stent device 20 would permit blood to flow through the parent vessel and across the arms 77 of those adjoining vessels at the bifurcation.

1. A stent device for introduction into and for treatment of a bifurcation aneurysm, comprising:
a radially expandable elongated wire mesh tube having a proximal end and a distal end, said distal end being expandable into a cone shaped portion of a larger diameter than said proximal end; and
a web of material arranged across said distal end of said stent device for sealing an aneurysm at a neck portion thereof.

2. The stent device as recited in claim 1, wherein said web is foraminous.

3. The stent device as recited in claim 1, wherein said web in non-foraminous.

4. The stent device as recited in claim 1, including an arrangement of struts at said distal end of said tube.

5. The stent device as recited in claim 4, wherein said struts have an aneurysm neck-portion-engaging distal end.

6. The stent device as recited in claim 1. wherein said web has openings therethrough which are smaller than wall openings in said proximal end of said tube.

7. The stent device as recited in claim 1, wherein said web is comprised of a foldable film of polymer.

8. The stent device as recited in claim 1, wherein said web is radiopaque.

9. The stent device as recited in claim 1, wherein said web is nestable across a neck portion of an aneurysm to seal embolytic material therein.

10. The stent device as recited in claim 1, wherein said web is pierceable by a microcatheter.

11. A method of treating a bifurcation aneurysm comprising:
introducing a microwire into said aneurysm:
introducing a waffle-cone-shapable microcatheter into said aneurysm over said microwire;
expanding a distal end of said microcatheter into a tapered waffle-cone shape within said bifurcation aneurysm; and
pivoting outwardly an annular array of anchoring struts from a circumferential array thereof spaced proximately adjacent said distal end of said catheter so as to anchor said distal end of said microcatheter within said aneurysm.

12. The method of claim 11, including:
arranging a pierceable web across said distal end of said microcatheter.

13. The method of claim 11, including:
introducing an embolitic agent through said catheter and said web on said distal end of said catheter, distally beyond said anchoring struts and into said aneurysm.

14. The method of claim 12, wherein said pierceable web is foraminous.

15. The method of claim 12, wherein said pierceable web is a non-foraminous film, to provide a sealed distal end to permit an unencumbered aneurysm-entering distal motion of said catheter.

16. The method of claim 11, wherein said struts have an outer end of “ski-tip” shape, to facilitate anchoring said catheter within a neck portion of the aneurysm.

17. The method of claim 16, including:
forming said ski-tip end of said struts to extend separate and apart from said distalmost end of said microcatheter.

18. A stent device for introduction into and for treatment of a bifurcation aneurysm, comprising:
a radially expandable elongated wire mesh tube having a proximal end and a distal end, said distal end being expandable into a cone-shapeable portion of a larger diameter than said proximal end, said stent having foraminous wall portions with openings therein of a first opening size;
a pierceable web of material arranged across said distal end of said stent device for sealing an aneurysm at a neck portion thereof, said web having foraminous portions with openings therein of a second opening size, said second opening size being smaller than said openings of said first opening size, said web sealing said aneurysm.

19. The stent device as recited in claim 18, having a circumferential array of radially outwardly splayable anchoring struts, said circumferential array of struts disposed proximately adjacent a zig-zag distalmost wire wall portion of said cone-shapeable portion of said stent device.

20. The stent device as recited in claim 19, wherein said anchoring struts have a distalmost tip portion which is free of attachment to said zig-zag distalmost wire wall portion of said stent device and said web is foraminous.
